(19) United States
(12) Patent Application Publication
(10) Pub. No.: US 2010/0099074 A1
(43) Pub. Date: Apr. 22, 2010

(54) COLLECTION DEVICE AND METHOD FOR STIMULATING AND STABILIZING A BIOLOGICAL SAMPLE

(76) Inventors: Garry Nolan, San Francisco, CA (US); Matthew Hale, Palo Alto, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/507,719

(22) Filed: Jul. 22, 2009

Related U.S. Application Data
(60) Provisional application No. 61/083,854, filed on Jul. 25, 2008.

(51) Int. Cl. A01N 1/02; C12M 1/00

(52) U.S. Cl. 435/2; 435/307.1

(57) ABSTRACT

A collection device and methods for stimulating and stabilizing a biological sample are provided. Aspects of the device include an evacuated sample collection container having stimulating and stabilizing additives contained in separate internal chambers of the container. The internal chambers are arranged to facilitate stimulation upon collection of a sample, followed by stabilization of the sample after a desired period of time in the same sealed container. Also provided are methods for use of the subject device. The device and methods find use in a variety of different applications, including the collection and analysis of clinically relevant samples, such as whole blood and other body fluids, obtained directly from a patient.

### CROSS-REFERENCE TO RELATED APPLICATIONS

Pursuant to 35 U.S.C. §119 (e), this application claims priority to the filing dates of U.S. Provisional Patent Application Ser. No. 61/083,854 filed Jul. 25, 2008; the disclosure of which application is herein incorporated by reference.

### ACKNOWLEDGEMENT OF GOVERNMENT SUPPORT

This invention was made with a Graduate Research Fellowship held by inventor Matthew Hale, awarded by the National Institute of Health, and Grant No. N01-HV-28183, awarded by the National Heart, Lung and Blood Institute. The Government has certain rights in this invention.

### INTRODUCTION

Conventional diagnostics and most clinical research have been focused on measuring the biological state of a sample. In recent years there has been growing interest in exposing patient material to stimulatory agents, such as immunomodulatory cytokines, and measuring the changes in numerous cellular parameters, especially intracellular signaling and genome-wide transcription. Several studies have shown that interrogating patient samples with stimuli reveals otherwise invisible biological states that have substantial clinical and diagnostic value.

A significant obstacle is that most facilities that routinely collect biological samples from patients lack the ability to carry out well-controlled stimulation experiments. Specific problems include the delivery of a precise amount of stimulus to the blood, allowing the stimulation to proceed for a defined period of time, and stabilizing the sample for later assessment of signaling state or transcript abundance.

The sample collection containers represent part of the problem. Sample collection containers have been in use for many years for collecting and storing blood and other body fluids. Typically, the collection containers are glass or plastic having a resilient stopper. Blood collection tubes are available where the tube is evacuated to draw a defined volume of blood into the tube (e.g., U.S. Pat. No. 3,848,579). The tubes can have various additives contained therein, for preparing the blood sample for a particular test. A common additive is an anticoagulant such as ethylenediaminetetraacetic acid (EDTA), buffered citrate, or heparin in a dried state (e.g., U.S. Pat. No. 6,428,527). Other tubes contain one or more fixatives that stabilize nucleic acids in the sample (e.g., U.S. Pat. No. 6,602,718). While such containers aid in the collection, or the collection and stabilization of samples directly from patients, the samples either require precise handling and manipulations for stimulation testing as noted above, or when immediately stabilized by a fixative, stimulation experiments on such samples are of little value.

As such, patient materials are currently either shipped live to laboratories capable of carrying out the stimulation assays of interest or are cryopreserved prior to shipping. Unfortunately, proper cryopreservation is beyond the capabilities of many facilities and it can be difficult to ship certain samples live. This is particularly difficult for blood samples positive for HIV or other infectious agents. Moreover, both of these approaches have been shown to induce undesirable changes in intracellular signaling and gene transcription, and yield results that may not reflect the true biology of the system.

Accordingly, there is a need for sample collection containers and methods that are convenient to use, safely contained, and facilitate improved handling and analysis of biological samples, such as whole blood and other body fluids, drawn directly from a patient. The present invention addresses these and other needs.

### SUMMARY

The present invention is directed to a device and methods for collecting, stimulating and stabilizing a biological sample from a patient, such as whole blood and other body fluids.

The device includes a sample collection container having arranged therein: (i) a partition defining and fluidly separating first and second internal chambers, where the partition includes a valve in fluid communication with the first and second internal chambers, and where the first chamber is positioned in the container for collecting a biological sample; and (ii) at least one stimulating agent contained within the first chamber in an amount effective to stimulate a biological sample, and at least one stabilizing agent contained within the second chamber in an amount effective to stabilize the biological sample. As the subject device is applied, the methods involve: (i) collecting a biological sample into the first chamber so as to immediately expose the biological sample to the stimulating agent; (ii) stimulating the biological sample in the first chamber for a desired period of time so as to form a stimulated biological sample; and (iii) stabilizing the stimulated biological sample immediately after the desired period of time by opening of the valve and mixing contents of the first and second chambers.

Also provided are kits and systems of the present invention. The subject device and methods find use in a variety of different applications, including research and development, clinical research, and diagnostic interrogation in general of a variety of biological samples. The device and methods of the invention are particularly useful for collecting and preparing a biological sample, such as blood, for assaying cellular signaling and/or transcriptional response to stimulation. An exemplary application, illustrating a significant advantage of the device and methods of the invention, is the collection of a predetermined volume of whole blood directly from a patient in an evacuated sample collection container of the invention that facilitates the automated stimulation and stabilization of the sample in the same sealed collection container.

### BRIEF DESCRIPTION OF THE FIGURES

FIG. 1 is a cross-sectional side view of a device of the invention.

FIG. 2 is a cross-section side view of components of a device of FIG. 1. Panel A depicts a closure member, and components internal to a device of FIG. 1. Panel B depicts an empty container in to which the internal components are arranged.

FIG. 3 is an expanded view of a plunger member of a device of FIG. 1.

FIG. 4 is a top angle view, a top view, a side view of a spring retention member of a device of FIG. 1.

FIG. 5 is a cross-section side view illustrating an operation of a device of FIG. 1.

FIG. 6 is a cross-section top view of a device of the invention.

FIG. 7 is a cross-section side view of the device of FIG. 6.

### DETAILED DESCRIPTION

The present invention is directed to a device and methods for collecting, stimulating and stabilizing a biological sample. The device of the invention includes a sample collection container having stimulating and stabilizing additives contained in fluidly separate chambers of the container. A first chamber in the container defines a sample collection and stimulating chamber, and a second chamber in the container defines a sample stabilizing chamber. The first and second chambers are fluidly separated by a partition having a valve member to provide and control fluid communication therein between.

Upon collection of a biological sample in the first chamber, the sample is immediately stimulated by the stimulating agent present in that chamber. After a desired period of time, the valve member can be opened to permit mixing of the contents of the first and second chambers. Mixing of the contents of the first and second chambers generates a stimulated and stabilized sample. As the sample on collection can be immediately stimulated for a defined period of time, and then immediately thereafter stabilized to fix and preserve the stimulated biological state inside the same sealed container, the device and methods of the invention facilitate a highly controllable sample collection process that is convenient to use, safely contained, and ready for subsequent analysis without exposing the user to potentially hazardous agents in the sample.

An exemplary application is the collection of a predetermined volume of whole blood directly from a patient in an evacuated sample collection container of the invention, where the sample collection container facilitates the automated stimulation and stabilization of the sample in the same sealed collection container. As such, the present device and methods are particularly well suited for collecting blood and stimulating it safely, within the sealed body of the apparatus, with high precision and consistency even in remote testing locations where limited tools are available for conducting stimulation experiments. After allowing the stimulation to proceed for a pre-specified duration, the device can automatically stabilize the sample for immediate testing, storage or shipment to a location capable of assaying cellular signaling and/or transcriptional response to stimulation. Accordingly, the device and methods of the invention represents a new class of research and diagnostic tool for measuring not just the steady state, but response to stimulation of a clinically relevant biological sample.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In addition, many modifications may be made to adapt a particular situation, material, composition of matter, process, process step or steps, to the objective, spirit and scope of the present invention. All such modifications are intended to be within the scope of the claims made herein.

In this specification and the appended claims, the singular forms “a,” “an” and “the” include plural reference unless the context clearly dictates otherwise. Conversely, it is contemplated that the claims may be so-drafted to exclude any optional element. This statement is intended to serve as antecedent basis for use of such exclusive terminology as “solely,” “only” and the like in connection with the recitation of claim elements or by use of a “negative” limitation

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Also, it is contemplated that any optional feature of the inventive variations described herein may be set forth and claimed independently, or in combination with any one or more of the features described herein.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All existing subject matter mentioned herein (e.g., publications, patents, patent applications and hardware) is incorporated by reference herein in its entirety. The referenced items are provided solely for their disclosure prior to the filing date of the present application. Nothing herein is to be construed as an admission that the present invention is not entitled to antedate such material by virtue of prior invention.

In further describing the subject invention, the subject device and methods are described first in greater detail, followed by a detailed description of exemplary embodiments, and then a review of various kits and systems that may find use with the subject device, as well as a discussion of various representative applications and advantages of the subject device and methods.

### Device and Methods

As summarized above, the subject invention provides a device and methods for collecting, stimulating and stabilizing a biological sample. Aspects of the device include a sample collection container having stimulating and stabilizing additives contained in separate internal chambers of the container. The internal chambers are arranged to prevent coagulation and facilitate stimulation upon collection of a sample, followed by stabilization of the sample after a desired period of time in the same sealed container.

In one embodiment, an apparatus for collecting, stimulating and stabilizing a biological sample is provided. The apparatus comprises a container having a side wall, a bottom wall, and a closure member defining an internal compartment. The internal compartment has arranged therein a partition defining and fluidly separating first and second chambers in the internal compartment. The first chamber is positioned in association with the closure member to receive the biological sample, and the partition includes a valve in fluid communication with the first and second chambers. The first chamber contains at least one stimulating agent in an amount effective to stimulate a biological sample, and the second chamber contains at least one stabilizing agent in an amount effective to stabilize the biological sample.

The stimulatory additive facilitates the stimulation of a particular biological response of the sample. The stabilizing additive facilitates the capturing and preservation of the stimulated state of the sample. The stimulating and stabilizing additives may be present as liquids or solids, where both types of additives may be liquid or solid, or one may be liquid and the other solid. For liquid forms of the additives, the liquid may be present in the form of a solution, suspension or dispersion. For solid formats, the solid in the form of a powder, such as a lyophilized material applied or sprayed into the chamber, or coating beads or integrated into a bead matrix, for example to provide a faster, more uniform dispersion.

As applied, upon collecting a sample in the first chamber (i.e., the collection and stimulating chamber), the sample is immediately exposed to the stimulating agent contained therein. After a period of time sufficient to cause the desired sample stimulation, the valve is opened to allow the sample fluid in the first chamber to admix with the stabilizing additive of the second chamber (i.e., the stabilizing chamber) so as to expose the stimulated sample to the stabilizing agent. Fluid exchange through the open valve can be promoted manually or automatically, or both, depending on preference and/or given end use. As such, the stimulated sample is fixed or otherwise arrested in its stimulated state. The stimulated and stabilized sample can be analyzed immediately, or it can be stored or shipped for later analysis.

The biological sample can be a body fluid or solid biopsy obtained from a patient. In one embodiment, the biological sample is whole blood. Other biological samples include cell-containing compositions such as red blood cell concentrates, platelet concentrates, leukocyte concentrates, plasma, serum, urine, bone marrow aspirates, cerebral spinal fluid, tissue, cells, and other body fluids. Also of interest are solid tissue samples, e.g., easily dissociated biopsies.

The device and methods of the invention are particularly suited for genomic and proteomic analysis of a biological sample collected directly from a patient, such as measuring changes in numerous cellular parameters, especially intracellular signaling and genome-wide transcription. An advantage of interrogating patient samples with stimulatory agents, such as immunomodulatory cytokines, is that it reveals otherwise invisible biological states that have substantial clinical and diagnostic value.

To maximize certain benefits of the present invention and obtain the most physiologically and clinically relevant results, the biological sample is stimulated with a controlled dose of the stimulating agent immediately after being drawn from the patient in the first chamber, and after a precise predetermined time interval, the signaling and transcription patterns are rapidly fixed in their stimulated state by one or more stabilizing agents contained in the second chamber. For use as a diagnostic, automated devices of the present invention are of particular interest, where the stimulation and fixation can be carried out automatically by the device, without supervision after initial drawing of the sample from the patient, and take place inside the sealed interior of the container to prevent exposure to pathogens that may be present in the patient sample.

As noted above, the valve can be automated or manually induced to open after a defined period of time. Opening of the valve places the first and second chambers in fluid communication and induces fluid mixing therein between. Mixing of the contents of the first and second chambers can be automated or manually induced as well. For example, the valve can be opened manually, such as by inserting a needle into the sealed container through a self-sealing closure member, or by non-invasively displacing or otherwise rupturing the partition to break the fluid seal and create an opening that places the first and second chambers in fluid communication. A particular embodiment of interest is an apparatus for collecting a biological sample that, upon collection, automatically stimulates and then stabilizes the sample in the same sealed container.

In certain embodiments, the partition is a plunger slidably supported in the internal compartment, where the plunger is connected to a plunger actuator in the internal compartment. In one embodiment, plunger actuator is a spring-loaded plunger actuator comprising a spring member, and a spring retention member associated with the internal compartment of the container, where the spring member exerts a force on the spring retention member and plunger so as to exert a spring force biasing the plunger towards the second chamber of the container. The plunger is composed of a material capable of maintaining a substantially fluid seal with the side wall of the internal compartment, such as a resilient material suitable for this purpose. A related aspect is where first chamber has an internal pressure that is less than atmospheric pressure, and the second chamber encloses a fluid exerting a fluid force on the plunger in opposition to the spring force. Another related aspect is where the internal pressure of the first chamber is specified to draw a predetermined volume of said biological sample directly into the first chamber.

In some embodiments, the valve is a time delay valve, which can be opened after a desired period of time upon receipt of a biological sample in the first chamber. By “desired period of time” is meant that the biological sample and stimulating agent in the first chamber is admixed with the stabilizing agent in the second chamber anywhere from immediately after to up to 5 hours or more, e.g., 10 hours, 15 hours, 20 hours or more, after the biological sample is received in the first chamber of the device. In general, stimulation of the sample in the first chamber ranges in increments of seconds from about 1 minute to 12 hours, and usually from about 5 minutes to about 6 hours, depending on the sample and particular assay of interest.

In a related embodiment, the time delay valve is opened following a predetermined period of time after receiving the biological sample in the first chamber. The predetermined period of time may be readily chosen depending on a particular given end use, and is generally between about 0.01 to 12 hours, and usually about 5 minutes to about 6 hours, depending on the sample and particular assay of interest as noted above.

In some embodiments, the time delay valve comprises an electric valve actuator electrically coupled to a time delay circuit and a power source for controlling the electric valve actuator. In certain embodiments, the time delay valve is a removable plug disposed in a passage of the plunger, where the removable plug may be affixed to the passage by a removable material. Of specific interest is an embodiment where the removable material is wax, the electric valve actuator comprises an electrical heating element, and the wax is in heating communication with the heating element.

In certain embodiments, the time delay circuit and power supply are disposed in the second chamber in association with the bottom wall of the container. The time delay circuit and power supply may also form the bottom wall, for example, as a sealing means of a tube having two open ends.

Another embodiment is where time delay valve is operatively connected to a sensor for detecting the presence of the biological sample in the first chamber, and for opening of the time delay valve after a specified period of time upon the detecting, where the sensor is electrically coupled to the time delay circuit. Of specific interest is a sensor which comprises a switch having an off state and an on state controlled by electrical conductivity of the first chamber. In one embodiment, the sensor is in an off state when there is no biological sample in the first chamber, and the switch is in an on state when the sample is present. When the switch is off, the timer means of the time delay circuit is idle. When the switch is on, the timer is armed. Once armed, the timer monitors a predetermined passage of time. When the timer senses that the predetermined passage of time has passed, the timer engages a second switch of the time delay circuit that controls and causes the electric valve actuator to open the valve. The sensor may be positioned in various locations in the first chamber, for example, associated with the plunger, interior side wall, or freely disposed therein.

The container can be made of glass, plastic or other suitable materials. Plastic materials can be oxygen impermeable materials or contain an oxygen impermeable layer. Alternatively, the container can be made of a water and air permeable plastic material. Transparent materials are of particular interest, such as transparent thermoplastic materials like polycarbonates, polyethylene, polypropylene, polyethylene-terephalate. The container also has a suitable dimension selected according to the required volume of the biological sample being collected. In one embodiment, container has a tubular shape with an axial length of about 60-mm to 130-mm and a diameter of about 10-mm to 18-mm. A container that is a tube having an axial length selected from about 75-mm and 100-mm millimeters and a diameter selected from about 13-mm to 16-mm are of particular interest. Plastic containers with the above features are of particular interest.

The closure member is made of a resilient material to provide a seal for retaining the biological sample in the container. Of specific interest is a closure made of a resilient material capable of maintaining an internal pressure differential less than atmospheric and that can be pierced by a needle to introduce a biological sample into the container. Suitable materials for closure include, for example, silicone rubber, natural rubber, styrene butadiene rubber, ethylene-propylene copolymers and polychloroprene. Thus in certain embodiments, the closure member is a septum pierceable by a cannula. The closure may also provide convenient access to a biological sample within the container, as well as a protective shield that overlies the closure. As such, the closure may further include a removable cover, such as a threaded or snap-on cap or other suitable member that can be applied over the outside of the closure for various purposes. For instance, a threaded cap can be screwed over the closure after the sample collection to provide a second seal and further increase user safety. Any component of the device can be color coded, labeled, or otherwise tagged or marked for easy identification.

The device as assembled can be provided to maintain an internal pressure differential between atmospheric pressure and is at a pressure less than atmospheric pressure. The pressure can be selected to draw a predetermined volume of a biological sample. Typically, the biological sample is drawn into the first chamber by piercing a closure comprising a resilient material with a needle or cannula, such as is typical for known evacuated sample containers for drawing blood. An example of a suitable containers and closures are disclosed in U.S. Pat. No. 5,860,937, which reference is incorporated in its entirety.

In one embodiment, the internal pressure of the container of the device and the volume of stabilizing additive in the second chamber are selected to provide the necessary concentration of the stabilizing agent for the volume of the biological sample collected. One aspect of interest is where the internal pressure of the container is selected to draw a predetermined volume of about 2.0 ml to about 10 ml of biological sample into the first chamber, and more particularly, about 2.5 ml to about 5 ml into the first chamber of the device. In certain embodiments, the selected draw volume for the first chamber and the volume of the stabilizing agent fluid in the second chamber are about equal. In alternative embodiments, the container can have an internal pressure at substantially atmospheric pressure.

In one embodiment, the container is made of a plastic that is water and gas permeable. Water loss by evaporation of the agent through the permeable of the container increases the concentration of the stabilizing agent and decreases the pressure within the container. The diffusion of oxygen through the wall of the tube has the effect of decreasing the vacuum in the container. The water and oxygen permeability properties of the container are selected to maintain the desired pressure differential within the container for the desired shelf life of the container. The shelf life is optimized by balancing the oxygen permeability with the water loss, preferably, the container has a life of about one year.

In certain embodiments, the first chamber has a pressure less than atmospheric pressure for drawing a predetermined volume of said biological sample in the container, the stabilization agent is present in the first chamber as a powder, and the second chamber encloses a fluid comprising the stabilizing agent. The internal compartment of the device is sterile for most applications. The second chamber may also include a sealable port in communication with an exterior of the side wall for receiving the stabilizing agent into the second chamber.

In the subject device, an effective amount of a stimulating agent is provided in the first chamber, and an effective amount of a stabilizing agent is provided in the second chamber. By “effective amount” is intended to mean a sufficient amount of the compound to provide the desired utility. For instance, for intracellular signaling or gene induction, the effective amount for the stimulating agent is the amount which elicits or is calibrated to elicit a useful response compared to controls (e.g., increase or decrease in phosphorylated protein content, increase or decrease in post-translational modification of specific proteins, increased mRNA abundance for a gene etc.). An effective amount for the stabilization agent can also be ascertained in this way, for instance, by determining the stability of a desired species in the samples relative to a control. As such, an appropriate effective amount may be determined by one of ordinary skill in the art using only routine experimentation.

The stimulating agent is normally provided as a powder and is present in an amount to affect at least one cellular activity that alters the cellular steady state (i.e., induced or reduced in abundance or activity). As such, the stimulating agent can comprise various compounds and formulations, such as intracellular signal inducing and immunomodulatory agents. Examples include small molecule drugs as well as peptides, proteins, lipids carbohydrates and the like. Of particular interest are compounds such as type I interferons (e.g., IFN-alpha, IFN-beta), interleukins (e.g., interleukin-2 (IL-2), IL-4, IL-6, IL-7, IL-10, IL-12, IL-15, IL-21), tumor necrosis factor alpha (TNF-{alpha}), gamma interferon (IFN-{gamma}), transforming growth factor β, and the like. In some embodiments, the stimulating agent comprises an immunomodulatory cytokine, such as immunomodulatory cytokines represented by interferons, interleukins, and chemokines among others. Interferon alpha is of specific interest.

The stabilizing additive is typically an aqueous solution, suspension or dispersion of at least one active stabilizing agent that is included in the device pre-filled in the second chamber. The concentration of the stabilizing agent present in the second chamber is adjusted to account for dilution upon mixing of the contents from the first and second chambers as noted above. Once the contents of the first and second chambers are combined to stabilize the stimulated biological sample, the stabilizing agent employed in the second chamber is diluted from the added volume of the stimulated sample. As such, the concentration of the stabilizing additive present in the second chamber before mixing is provided to take into account this dilution.

In certain embodiments, the stabilizing agent comprises an agent selected from a fixative, a cell lysis buffer that may specifically lyse erythrocytes, and mixtures thereof. Examples of fixatives include organics such as formaldehyde, formamide, dimethylsulfoxide, ethylene glycol, and diethylene glycol. Examples of cell lysis agents include saponin, detergents, cationic compounds, chaotropic salts, organic solvents such as phenol, chelating agents and the like. Hypotonic formulations aid in erythrocyte lysis. The concentration of cell lysis agents is adjusted for a given end use. When present at lower concentrations, cell lysis may be suboptimal. At higher concentrations, undesirable cellular disruption may occur. Routine empirical approaches can be carried out to determine the preferred route in each instance.

The stabilizing agent is generally selected based on a preference to carry out proteomic or genomic analysis of a treated sample post-stimulation/stabilization. For instance, the interest in developing stimulation assays as diagnostics or for research purposes is broadly separated into those that wish to focus on the biology of the sample at the protein level (proteomics: intracellular flow cytometry, Western blots etc.) and those that wish to focus on the biology of the sample at the nucleic acid level (genomics: microarrays, PCR etc.). The device and methods of the invention can be applied to meet both needs. Specifically, the device and method may employ different stabilization solutions such as one that stabilizes proteins and intracellular signaling, or one that stabilizes nucleic acid species.

For proteomic applications, the stabilizing agent is one that stabilizes proteins and intracellular signaling. Of particular interest are stabilizing agents that lyse erythrocytes but not leukocytes and preserve cell surface antigens while arresting at least one cellular process selected from protein synthesis, protein degradation, RNA synthesis, DNA synthesis, endocytosis, secretion, phosphorylation, dephosphorylation, ubiquitinization, methylation, and combinations thereof.

Of interest in certain embodiments are stabilizing agents that preserve cell surfaces suitable to permit single-cell sorting, such as fluorescence-activated cell sorting (FACS) or flow cytometry. Of interest is where intracellular phospho-specific antibody staining of the sample is analyzed by flow cytometry or FACS. FACS technology facilitates single-cell multiparametric analysis and sorting, based on physical properties of cells and/or their relative expression levels of specific protein or glycoprotein epitopes and metabolites. Recently, the use of fluorescent antibodies specific for unique phosphorylated epitopes—or “phospho-epitopes”—on proteins of interest has further extended the range of FACS analyses. This new application, dubbed “phospho-FACS”, has quickly become a tool of choice for delineating intracellular phosphorylation cascades. As such, the application of phospho-FACS to cellular subsets from blood or the periphery, whether frequent or rare, aids in the discovery of pathological biomarkers and therapeutic innovation. Because of its ability to generate single-cell data and resolve the heterogeneous mixtures of cells present in patient samples, the phospho-FACS technique features numerous advantages compared to other analytical methods for measuring signaling cascades.

In a particular embodiment, the biological sample is whole blood, and the stabilizing agent stabilizes the whole blood. Stabilizing agents of specific interest for this purpose includes aqueous solutions comprising a final concentration in the biological sample of about 0.1%-10% formaldehyde and about 0.003%-3% saponin, and more particularly about 1% formaldehyde and about 0.3% saponin.

Another aspect of the invention is where the stabilizing agent stabilizes nucleic acids from degradation, such as an agent that arrests transcription while stabilizing nucleic acids from degradation. The nucleic acid may be DNA or RNA, and varies species thereof. In certain embodiments, the nucleic acid is RNA, particularly mRNA and micro RNA. In a related embodiment, the stabilizing agent renders the RNA suitable for analysis by microarray hybridization or polymerase chain reaction. An additional aspect of the invention is where the stabilizing agent preserves cell surfaces suitable to permit single-cell sorting.

The stabilizing agents of specific interest are able to effectively stabilize DNA and RNA including mRNA and micro RNA. Examples of suitable stabilizing agents for stabilizing and preserving nucleic acids and preventing gene induction include cationic compounds, detergents, chaotropic salts, ribonuclease inhibitors, chelating agents, organic solvents such as phenol and Trizol®, and mixtures thereof, suitable ribonuclease inhibitors are high concentrations of exogenous transfer RNA or placental RNA inhibitor protein. Additional reagents of interest include urea, formaldehyde, guanidinium isothiocyanate, guanidinium hydrochloride, formamide, dimethylsulfoxide, ethylene glycol and tetrafluoroacetate. Examples of stabilizing agents of particular interest are those selected from: cationic compounds, detergents, chaotropic salts, ribonuclease inhibitors, chelating agents, and mixtures thereof. Of particular interest are such stabilizing agents in an aqueous solution or dispersion having a pH of about pH 2 to about pH 12. Various nucleic acid stabilizing agents are described in U.S. Pat. No. 6,602,718, which reference is incorporated herein in its entirety.

The stimulating and stabilizing agents may be provided in different forms or formulations in the device. By way of illustration, the agent can be admixed with conventional carriers and excipients (i.e., vehicles) and used in the form of powders, aqueous solutions, dispersions, bead dispersions (e.g., where the agent, such as stimuli and/or anticoagulant, is dried onto beads and/or impregnating a soluble bead matrix (˜2-100 μm beads dried down in a highly soluble substrate) to enhance the solubility and consistency of dispersion of certain stimulatory and/or anti coagulation agents), gels, foams, tablets, capsules, elixirs, suspensions, syrups, wafers, and the like. A fluid or liquid composition will generally consist of a suspension, dispersion or solution of the active agent in a suitable liquid carrier(s), for example, water, ethanol, glycerine, sorbitol, non-aqueous solvent such as polyethylene glycol, oils or water, with a suspending agent, preservative, surfactant, wetting agent, or coloring agent. Alternatively, a liquid formulation can be prepared from a reconstitutable powder. For example, a powder containing active compound and a suspending agent can be reconstituted with water to form a suspension or dispersion. Accordingly, there are a wide variety of suitable stimulating and stabilizing formulations of the present invention.

In an exemplary embodiment, the device comprises an evacuated blood sample collection container of the invention in which the first chamber has an internal pressure less than atmospheric pressure and contains a stimulating agent in combination with an anticoagulation agent, and where the second chamber comprises a stabilizing agent comprising a fixative in an erythrocyte lysis buffer. As applied, the method involves drawing whole blood into a sample collection container of the invention directly from a patient, where the first chamber comprises a stimulating agent in combination with an anticoagulation agent, and where the second chamber comprises a stabilizing agent comprising a fixative cell lysis buffer. This aspect of the invention facilitates sample stimulation while preventing coagulation in a first step, and after a specified period of time, sample stabilization with a fixative in a second step, so as to allow later analysis of the changes induced by the stimuli. Examples of anticoagulation agents are those selected from ethylenediaminetetraacetic acid (EDTA), buffered citrate, and heparin (e.g., heparin sulfate). Examples of the stabilizing agent are those selected from a fixative, a cell lysis buffer, or mixtures thereof, with mixtures being of particular interest (e.g., aqueous solutions comprising a final concentration in the biological sample of about 0.1%-10% formaldehyde and about 0.03%-3% saponin, and more particularly about 1% formaldehyde and about 0.3% saponin).

In using devices of the invention, once a biological sample is obtained, stimulated and stabilized, it may be stored for a period of time and then further analyzed, as desired, or further analyzed immediately, as desired. Storage may be under any convenient conditions, including low temperature (e.g., frozen) conditions, and may last for an extended period of time, e.g., several days or longer, 1 week or longer, 1 month or longer, 6 months or longer, etc. Further analyzing of the sample may occur at a location, e.g., laboratory, remote from the location at which the sample was obtained, e.g., in a different room of a building, in a different building, in a different town, in a different state or even in a different country. Accordingly, certain embodiments include transporting the sample from the first location at which the sample was obtained to a second location at which the sample is further analyzed, where transportation may include ground or air transportation.

A variety of different further analyses may be performed on the sample following stimulation and stabilization. Of interest in certain embodiments are the analysis protocols described in United States Provisional Application Publication Nos.: 20070196870 titled “Methods and compositions for detecting receptor-ligand interactions in single cells”; 20070009923 titled “Use of bayesian networks for modeling cell signaling systems”; 20060073474 titled “Methods and compositions for detecting the activation state of multiple proteins in single cells”; and 20050112700 titled “Methods and compositions for risk stratification”; the disclosures of which are herein incorporated by reference.

### Exemplary Device and Methods

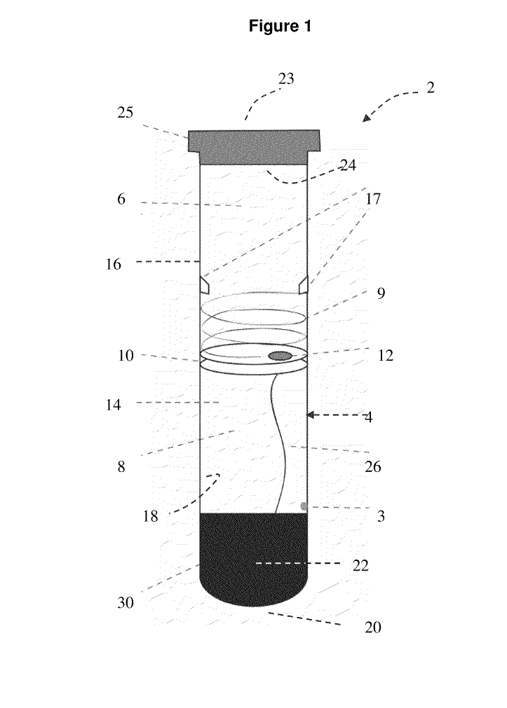

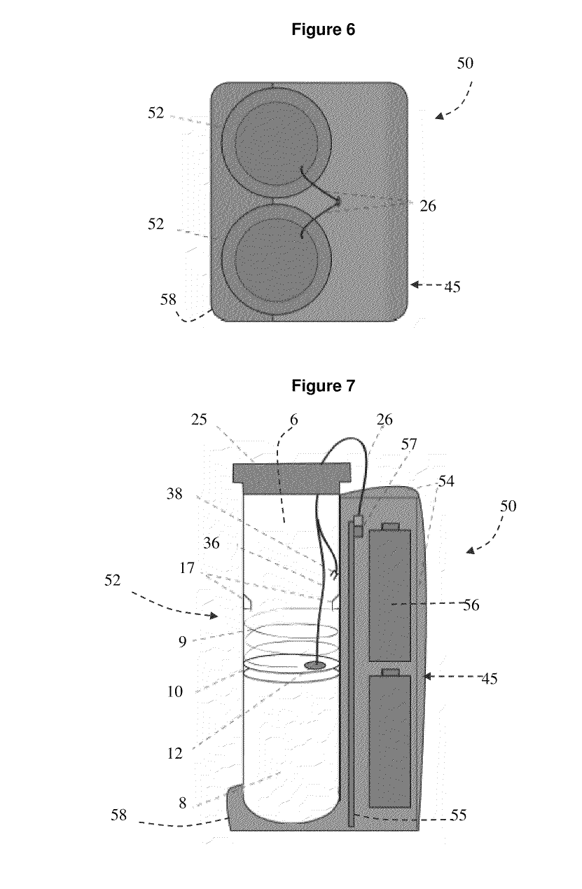

As discussed above, a featured embodiment is a device suitable for drawing a body fluid sample, such as whole blood, directly from a patient for automated stimulating followed by stabilization of the sample immediately at the point of collection. Referring to the drawings in detail, wherein like numbers designate like parts, two main embodiments of the featured sample collection device are illustrated: an embodiment in which the power source and electronics are contained within the container, FIGS. 1-5; and an embodiment in which the power source and electronics are outside the container, FIGS. 6-7. Although the featured sample collection devices are particularly useful for automated stimulation and stabilization of biological samples, it will be appreciated that the design and safety features of the exemplified embodiments are applicable to other collection devices and methods described herein and are not intended to be limiting.

Referring initially to FIG. 1, a cross section view of the device is depicted, with the apparatus in a pre-loaded state, ready to receive and process a sample. The numeral 2 designates the sample collection device. The device 2 includes a container 4 defining a sample collection and stimulating chamber 6 and a sample stabilizing chamber 8, and a plunger 10 that defines and fluidly separates chambers 6 and 8. Valve 12 provides and controls fluid communication between chambers 6 and 8. Valve 12 is disposed in a passage of plunger 10, which is capable of transforming an input signal, such as an electrical signal, into motion to induce mixing of the contents of sample collection and stimulating chamber 6 and sample stabilizing chamber 8 when valve 12 is open. As also illustrated, container 4 is a tube defining an internal compartment 14 having a side wall 16 defining a side wall interior 18, a closed bottom end 20 defining a bottom end interior 22, and an open top end 23 defining a top end interior 24. Stimulating agent is disposed in chamber 6 in an effective amount to stimulate a biological sample received in this chamber. Stabilizing agent is disposed in chamber 8 in an effect amount to stabilize a stimulated biological sample received in chamber 6. Closure 25 is positioned at the open top end 23 to close container 4 and defines an access opening of sample collection and stabilizing chamber 6. Closure 25 is made of a resilient material suitable for forming a seal capable of effectively closing container 4 and retaining a biological sample in container 4. Container 4 includes a sealable port 3 for conveniently introducing stabilizing agent into sample stabilizing chamber 8, of which a heat sealable port is an example. Container 4 also is dimensioned for collecting, stimulating and stabilizing a suitable volume of a biological fluid.

FIG. 1 also illustrates an embodiment of a plunger actuator suitable for controlling plunger 10 and valve 12. Plunger 10 is slidably supported in internal chamber 14 in substantially sealing proximity to side wall interior 18. Spring member 9 is retained within container 4 by side wall interior 18 and opposing forces of spring retention member 17 and plunger 10. In certain embodiments, spring member 9 can be loaded to expand when disposed within sample collection and stimulating chamber 6, or loaded to contract when disposed within sample stabilizing chamber 8 along with spring retention means. As illustrated, the plunger actuator is a spring-loaded plunger actuator assembly having spring member 9, and spring retention member 17 fixably mounted to side wall interior 18 of chamber 6. Spring member 17 is loaded to exert a force on spring retention member 17 and plunger 10 so as to exert a spring force biasing plunger 10 towards the second chamber 8. Valve 12 is closed and sample stabilizing chamber 8 encloses a sample stabilizing fluid exerting a fluid force on plunger 10 in opposition to the spring force.

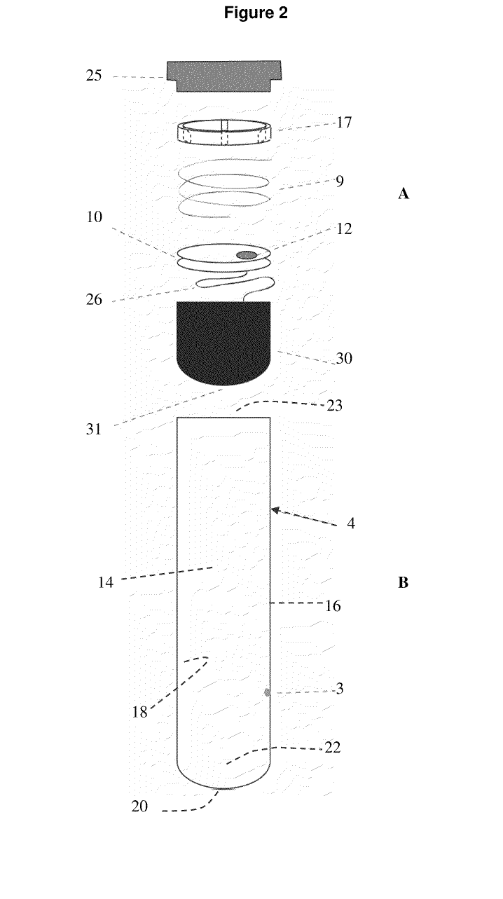

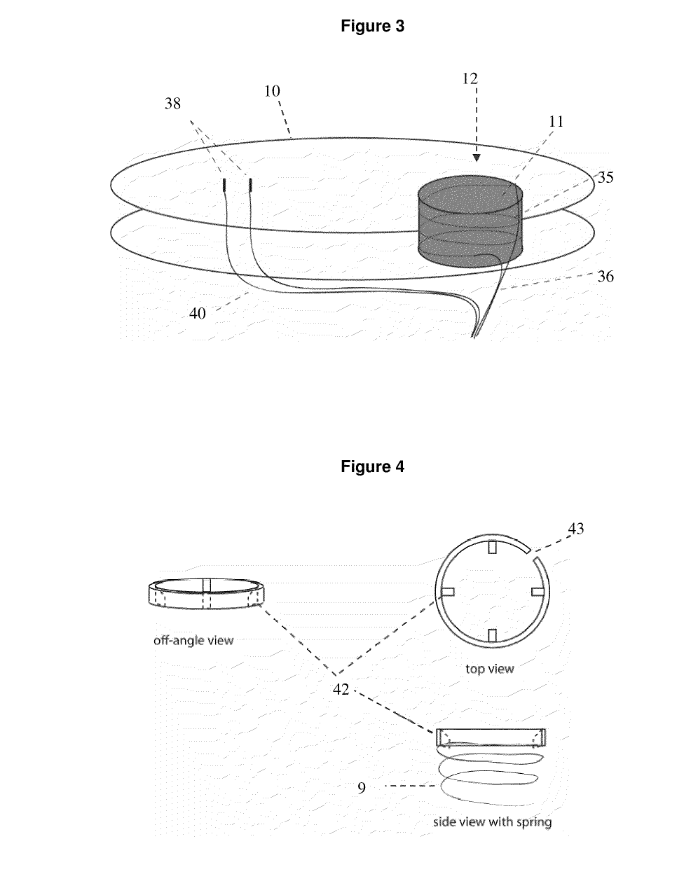

Valve 12 in this example is configured as a time delay valve capable of being automatically opened after a predetermined period of time following collection of the biological sample in sample collection and stimulation chamber 6. Valve 12 is controlled and electrically connected to a triggerable time delay circuit and power source 30 by control wires 36 (FIG. 3). An adhesive means 31 (FIG. 2) may be included to adhesively secure time delay circuit and power source 30 to bottom end interior 22.

Referring to FIG. 3, an expanded view of one embodiment of plunger 10 and valve 12 is illustrated. Valve 12 is controlled by an electric valve actuator electrically coupled to time delay circuit and power source 30. The electric valve actuator includes electrical resistance heating element 35. Heating element 35 is in heating communication with a wax coated removable valve plug 11. Valve plug 11 is sealably disposed in a passage of plunger 10 by the wax. Heating element 35 forms a coil circumscribing valve plug 11 and is embedded in the wax, and is electrically connected to valve control wires 36. Valve control wires 36 are electrically connected to time delay circuit and power source 30. When current is applied by time delay circuit and power source 30, heating element 35 reaches a localized temperature sufficient to melt the wax. Valve plug 11 is under pressure from the stabilization agent fluid in chamber 8 so that when the wax melts, valve plug 11 is easily dislodged from the passage in plunger 10 by the difference in hydraulic pressure. After a period of time that is sufficient for valve 12 to open, the current to heating element 35 is discontinued (approximately 1 minute). Valve plug 11 can be any suitable material sufficient to form a fluid seal with the passage in plunger 10, of which examples include a plastic plug smaller in diameter than the passage but coated in a thin (˜2 mm) layer of wax that allows it to form a good seal. Different wax compounds have different melting temperatures, and can be selected for a given end use by routine procedures. Examples of heating element 35 include electrical resistance heating materials such as nichrome wire.

FIG. 3 further illustrates a particular embodiment of a sensor for detecting the presence of a biological sample in sample collection and stimulating chamber 8, and arming timer means of timer delay circuit and power source 30 for opening valve 12 after a desired period of time. Valve 12 is operatively connected to sensor 38, which is electrically coupled by arming wires 40 to time delay circuit and power source 30. Sensor 38 is an electrical switch means sealably passing through plunger 10, and having leads in electrical communication with the interior of sample collection and stimulating chamber 8. The presence of a sufficiently electrically conducting fluid such as blood in chamber 6 places sensor 38 switch in on-state to arm timer means of time delay circuit and power source 30.

A particular embodiment of spring retention member 17 is illustrated in FIG. 4. Off-angle and top views show spring retention teeth 42 capable of retaining spring member 9 within container 4. In certain embodiments, spring retention member 17 is made of the same material as container 4; for example, when container 4 is a plastic material, spring retention member 17 may be composed of the same material so that a solvating adhesive may be applied to fuse the two together at the point of assembly. Spring retention member 17 may also be formed by container 4, closure 25, or other spring retention means in interior compartment 14. The top view of spring retention member 17 illustrates a split ring design having gap 43 to provide a flush fit and proper adhesive contact with side wall interior 18 of container 4.

Referring FIGS. 6-7, an embodiment is illustrated in which time delay circuit and power supply 30 is positioned on the outside of container 4. External device 45 includes a time delay circuit in circuit board 55 and power source 56 for controlling one or more sample collection containers comprising device 52. External device 45 also includes rack 58 for reversibly receiving and retaining multiples of each device 52. FIG. 6 shows a top view of external device 45, which is shown retaining two separate units of each device 52, forming apparatus 50. FIG. 7 shows a side view of external device 45, which illustrates rack 58 retaining means for reversibly receiving and retaining device 52. Arming and control wires 26 controlling valve 12 in each device 52 allow for independent control of each device 52, are electrically coupled to circuit board 55 through a 4-pin connector 57, and sealably pass through closure 25 into an interior of sample collection and stimulation chamber 6 to expose sensor 38 leads in this chamber. Removable panels 54 provide access to circuit board 55 and power source 56.

External device 45 can independently control each device 52 in a manner in which one device 52 contains a stimulation agent and the other does not (experimental control sample). External device 45 may further include programmable means in circuit board 55 to independently specify and track the stimulation time for each device 52 and to independently initiate stabilization after a pre-specified stimulation time. An advantage of external device 45 is that multiples of device 52 can be independently controlled. Also, because external device 45 does not come into direct contact with the biological sample, the device can be reused. The multiunit capacity, reusability, programmable nature, and size of an apparatus exemplified by external device 45 embody additional benefits.

External device 45 can be externally powered or contain an internal power source. External device 45 can also include a heating unit capable of maintaining the incubation temperature of each device 52. Examples of the heating unit include heating element means positioned in heating communication with each device 52, the heating element being electrically connected to circuit board 55 and power source 56. As illustrated, external device 45 includes an internal power source and heating unit sufficient to maintain the temperature of the biological sample in each device 52 at physiological temperature (37° C.) for an extended period of time (e.g., an internal power source of 4 AA batteries permits temperature control for up to 6 hours). This is important for longer stimulation experiments that are of particular interest to investigators focused on transcriptional responses to stimulation. An example of a heating unit is one formed by a heating element jacket positioned externally and in heating communication with each device 52, the heating element jacket being electrically coupled to and controlled by circuit board 55 and power source 56. An example of a suitable heating element jacket is one composed of a coil of nichrome wire wrapped around the outside of each device 52. A thermocouple inside each device 52 can be included to sense the temperature of the biological sample, where the thermocouple is electrically connected to circuit board 55 having means to provide and control a negative feedback loop to maintain a desired temperature of the biological sample.

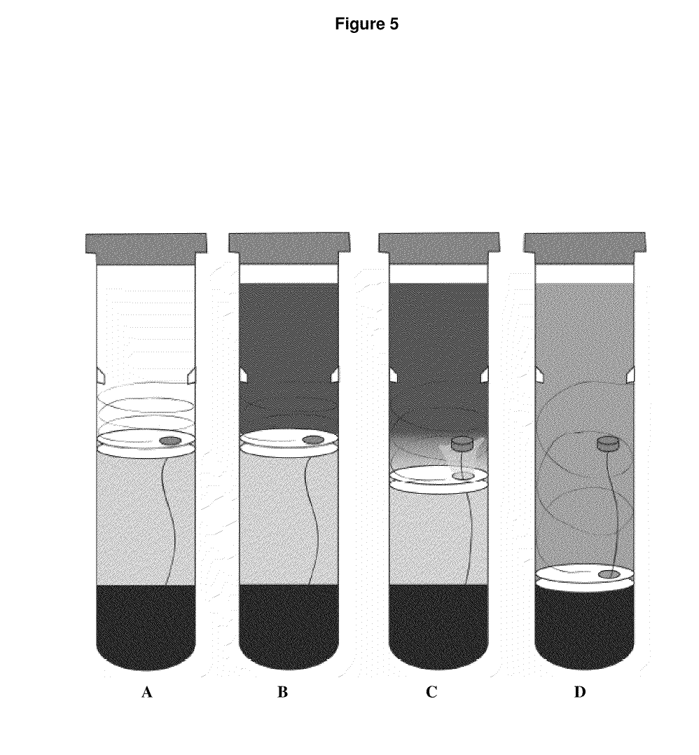

A diagram of the operation of one embodiment of device 2 is illustrated by reference to FIG. 5. In FIG. 5—panel A, device 2 is ready for collection. In FIG. 5—panel B, the biological sample is received in sample collection and stimulation chamber 8. In FIG. 5—panel C, valve 12 is opened and the fluid contents of the stabilization chamber 8 flows through as plunger 10 descends driven by spring member 9. FIG. 5—panel D shows the biological sample mixed with the fluid contents of sample stabilizing chamber 8. The stabilizing solution in the upper chamber is under pressure from a moderately powerful steel spring applying a downward force (e.g., ˜2 kilograms) on the plunger (FIG. 5—panels A and B). Like most liquids, the stabilizing solution on the other side of the plunger resists compression and hydrostatic pressure opposes the force of the plunger and maintains static equilibrium until the valve opens. When the valve opens, the difference in hydraulic pressure between the upper chamber and the lower chamber (generated by the spring means) allows the stabilizing solution to dislodge the valve plug and flow rapidly into the upper chamber containing the stimulated sample (FIG. 5—panel C). The difference in hydraulic pressure coupled with the smaller diameter of the valve opening relative to the plunger ensures that the stabilizing solution enters at a fast rate, ensuring significant mixing. As the stabilizing solution flows out of the lower chamber, the spring displaces the plunger downward maintaining the pressure gradient. This continues until the plunger reaches the bottom of the lower chamber at which point all of the stabilizing solution has been displaced into the upper chamber. The plunger displaces the entire volume of the lower chamber such that effectively only a single chamber remains in the apparatus containing the biological sample thoroughly mixed with the stabilizing solution (FIG. 5—panel D). Because the plunger increases the volume of the upper chamber at the same rate that the volume in the lower chamber is displaced, there is no net increase in pressure inside the container, and no risk of the closure member being loosened or ejected because the pressure inside the upper chamber never exceeds that of the atmosphere at the time the sample was drawn. The container can then be transferred to a freezer for storage and or is shipped cold to a laboratory wherever analysis of the sample takes place.

In describing a specific embodiment illustrated in FIG. 5, reference is made to another specific embodiment of the invention in which device 2 is an evacuated blood collection container where: (i) the biological sample is whole blood drawn directly from a patient; (ii) closure 25 is a conventional self-sealing rubber stopper; (iii) sample collection and stimulation chamber 6 comprises an effective amount of an immunomodulatory cytokine (e.g., interferon alpha) as the stimulating agent in combination with an anticoagulant (e.g., heparin sulfate); (iv) stabilizing chamber 8 contains an aqueous solution comprising a fixative-cell lysis buffer (e.g., 2% formaldehyde and 0.6% saponin) suitable for subsequent single-cell sorting (e.g., phosphor-fluorescence-activated cell sorting (phosphor-FACS)) and proteomic analysis of intracellular signaling of the processed sample; and (v) container 4 is dimensioned and evacuated to specify a blood draw volume approximately equal to the volume of the fixative-cell lysis buffer contained in stabilizing chamber 8.

As applied in this particular example, an embodiment of device 2 (e.g., FIG. 5—panel A) is employed to collect a blood sample directly from a patient. The sample collection and stimulation chamber 8 is evacuated to a pressure that allows a predetermined volume of blood (e.g., 5 ml) to be drawn in. The inside of the sample collection and stimulation chamber 8 is coated with anticoagulant (e.g., lyophilized heparin sulfate sprayed into the top chamber and dried down under low pressure). An immunomodulatory cytokine (e.g., interferon alpha) is also present in the sample collection and stimulation chamber 8 in lyophilized form as the stimulating agent (sprayed into the top chamber and dried down under low pressure).

As also illustrated in this example, blood is drawn into the sample collection and stimulation chamber 8 by piercing closure 25 with a hollow needle that is connected to disposable blood draw tubing connected to another hollow needle already inserted into the patient's vein following standard blood drawing protocols for evacuated blood collection containers (e.g., FIG. 5—panel B). The stimulating and anticoagulant compounds are extremely soluble in blood and are immediately solubilized upon contact with blood, and inverting the tube several times ensures proper mixing and begins stimulation of the cells in the sample. Inverting the device 2 to ensure proper mixing is a common practice for existing blood draw products. Immediately on collection of the blood sample in the sample collection and stimulation chamber 6, stimulation is initiated concurrent with detection and arming by sensor 38 of the timer means of time delay circuit and power supply 30 unit. Electrolytes in blood conduct electricity better than air so a large decrease in resistance across the leads of sensor 38 allows current to flow to and activate the timer means. During the time delay period (e.g., timer means preset at 15 minutes or other desired stimulation duration) the blood sample is exposed to the immunomodulatory cytokine stimulating agent. At the end of the time delay period, the time delay circuit and power supply 30 unit passes current through valve control wire 36 and heating element 35, valve 12 is opened, and the contents of the stabilizing chamber 8 is allowed to flow between chambers 6 and 8 as plunger 10 descends towards the bottom wall 20, causing the stimulated blood sample to admix with the sample stabilizing agent and initiate fixation and erythrocyte lysis of the stimulated blood (e.g., FIG. 5—panels C and D). 10 minutes after device 2 has mixed the contents of both chambers (e.g., FIG. 5—panel D), the device is refrigerated or frozen if the contents are processed and analyzed in a timely manner. The device can be stored briefly or for longer durations and the chemistry has been shown to suitably stabilize a sample for storage at −80° C. for at least one month before processing. What is important is that all samples in a study are treated in the same manner (i.e., if some samples are frozen at −80° C., then all of the samples in the study should be frozen at −80° C. though the duration of storage need not be the same). Processing of samples can be carried out by washing the stimulated/stabilized sample two times with phosphate buffered saline (PBS) and then, if required, erythrocyte debris removed by underlaying with bovine serum and centrifuging at ˜800 rpm. The cells are then washed again with PBS and the pellet resuspended in 2 ml of a solution of 80% methanol, 20% PBS pre-chilled to 4° C. The methanol fixed cells can then be stored at −80° C. or washed 2 times with staining media (PBS+0.5% bovine serum albumin) and analyzed by phospho-FACS (standard protocol).

### Kits & Systems

Also provided are kits and systems comprising a device of the invention that finds use in practicing the subject methods, as described above. For example, kits and systems that include a device for practicing the subject methods may comprise one or more components thereof, such as one or more of the stimulating agent and the stabilizing agent. The kits may also include a device that is pre-filled with such agents and ready-to-use, pre-filled with only one component, or provided separately as a blank device and reagents for assembly and application. In certain embodiments the kits include two or more containers, which may be the same or different with respect to the stimulating and/or stabilizing agents contained therein. For examples, kits of the embodiments may include a first container which is configured for nucleic acid analysis (such that it contains reagents chosen for nucleic acid analysis) and a second container which is configured for protein analysis (such that it contains reagents relevant for protein analysis), e.g., as described above. In certain embodiments, the kits include external reusable components, e.g., external power sources/controllers as described above.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., a diskette, a CD, etc., on which the information has been recorded. Yet another means that may be present is a website address which may be used via the internet to access the information at a remote site. Any convenient means may be present in the kits.

The term “system” as employed herein refers to a collection of a device of the invention, a stabilizing agent, and a stimulating agent, present in a single or disparate arrangement, that are brought together for the purpose of practicing the subject methods. For example, separately obtained device, stimulating agent, and stabilizing agent brought together to collect, stimulate and stabilize a biological sample, according to the present invention, are a system according to the present invention.

### UTILITY AND ADVANTAGES

The subject devices and methods find use in different applications, including research and development, clinical research, and diagnostic applications of a variety of biological samples. The device and methods of the invention have broad applicability for collecting, stimulating and stabilizing samples such as blood and other biological materials including leukocyte concentrates, cerebral spinal fluid, fluids from inflamed joints and tissues, plasma, serum, urine, bone marrow aspirates, and other tissues. This includes analysis of the impact of stimuli on clinically relevant samples for research and development, as well as for diagnosing a patient for baseline or disease-related indicators.

The device and method of the present invention have several advantages. The attendant benefits are particularly apparent for body fluid samples drawn directly from a patient, such as whole blood. For example, one of the advantages of the present invention is that it ensures consistency of stimulation and stabilization of the blood sample for analysis. Consistency of stimulation and stabilization of the sample for analysis have been the greatest obstacles to widespread adoption of stimulation assays for diagnostic or research purposes. Another advantage is that the sample is completely contained within the apparatus and the user is never exposed to an open container of live blood, thus minimizing the risk to the user. In contrast, existing methods for carrying out stimulation experiments require opening the collection container and manual transfer of liquids into the container or manual transfer of the blood into other containers.

In addition, at the time of shipping and processing, the sample will have already been stabilized with a solution that reduces or completely neutralizes any blood borne pathogens present in the sample. This minimizes the risk to those involved in shipping, receiving, and processing the sample (e.g., diagnostics for or studies of HIV or HCV). As existing methods require the shipping and handling of live blood or of blood products that have been cryopreserved (a technique that is not designed to reduce the risk of blood borne pathogens), the present invention substantially increases the safety of working with potentially infectious samples, especially if the donors are known to be positive for infectious agents.

It is evident from the above results and discussion that the subject invention provides for devices and methods of collecting, stimulating and stabilizing a biological sample, as well as analyzing the sample. It is also evident that the subject invention is not solely for the collection of blood, but other biological materials as well including leukocyte concentrates, cerebral spinal fluid, fluids from inflamed joints and tissues, plasma, serum, urine, bone marrow aspirates, and other tissues. As such, the subject invention finds use in a variety of different applications and represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. An apparatus for collecting, stimulating and stabilizing a biological sample, said apparatus comprising:
a container having a side wall, a bottom wall, and a closure member defining an internal compartment, said internal compartment having arranged therein: a partition defining and fluidly separating first and second chambers in said internal compartment, said first chamber positioned in association with said closure member to receive said biological sample, said partition having a valve in fluid communication with said first and second chambers; and
at least one stimulating agent contained within said first chamber in an amount effective to stimulate a biological sample, and at least one stabilizing agent contained within said second chamber in an amount effective to stabilize said biological sample.

2. The apparatus of claim 1, wherein said partition is a plunger slidably supported in said internal compartment.

3. The apparatus of claim 2, wherein said plunger is connected to a plunger actuator in said internal compartment.

4. The apparatus of claim 3, wherein said plunger actuator is a spring-loaded plunger actuator comprising a spring member, and a spring retention member associated with said internal compartment, said spring member exerting a force on said spring retention member and said plunger so as to exert a spring force biasing said plunger towards said second chamber.

5. The apparatus of claim 3, wherein said first chamber has an internal pressure that is less than atmospheric pressure, and said second chamber encloses a fluid exerting a fluid force on said plunger in opposition to said spring force.

6. The apparatus of claim 5, wherein said internal pressure is specified to draw a predetermined volume of said biological sample directly into said first chamber.

7. The apparatus of claim 5, wherein opening of said valve places said first and second chambers in fluid communication and induces fluid mixing therein between.

8. The apparatus of claim 3, wherein said valve is a time delay valve.

9-10. (canceled)

11. The apparatus of claim 8, wherein said time delay valve comprises an electric valve actuator electrically coupled to a time delay circuit and a power source.

12. The apparatus of claim 11, wherein said time delay valve is a removable plug disposed in a passage of said plunger.

13. The apparatus of claim 12, wherein said removable plug is affixed to said passage by a removable material.

14. The apparatus of claim 13, wherein said removable material is wax.

15. The apparatus of claim 14 wherein said electric valve actuator comprises an electrical heating element.

16. The apparatus of claim 15, wherein said wax is in heating communication with said heating element.

17. The apparatus of claim 16, wherein said heating element is a heating coil embedded in said wax.

18. (canceled)

19. The apparatus of claim 11, wherein said time delay circuit and power supply are disposed in said second chamber in association with said bottom wall.

20. The apparatus of claim 11, wherein said time delay circuit and power supply form said bottom wall.

21. The apparatus of claim 11, wherein said time delay valve is operatively connected to a sensor for detecting the presence of said biological sample in said first chamber and for opening of said time delay valve after a specified period of time upon said detecting.

22. The apparatus of claim 21, wherein said sensor is electrically coupled to said time delay circuit.

23. The apparatus of claim 22, wherein said sensor comprises a switch having an off state and an on state controlled by electrical conductivity of said first chamber.

24. The apparatus of claim 22, wherein said sensor is associated with said plunger.

25. The apparatus of claim 1, wherein said closure member is a septum pierceable by a cannula.

26. The apparatus of claim 1, wherein said second chamber includes a sealable port in communication with an exterior of said side wall to receive said stabilizing agent in said second chamber.

27-31. (canceled)

32. The apparatus of claim 1, wherein said stabilizing agent comprises a fixative.

33. The apparatus of claim 1, wherein said stabilizing agent comprises a cell lysis buffer.

34-53. (canceled)

54. An apparatus comprising a biological sample collection container, said container having a side wall, a bottom wall, and a closure member defining an internal compartment, said internal compartment having arranged therein:
a spring-loaded plunger actuator comprising a spring member, a plunger slidably supported in said internal compartment, and a spring retention member associated with said internal compartment, said spring member exerting a force on said spring retention member and said plunger so as to exert a spring force on said plunger, said spring-loaded plunger actuator positioned in said internal chamber so as to bias said plunger towards said bottom wall; and
first and second chambers defined and fluidly separated by said plunger, said first chamber positioned in association with said closure member for receiving a biological sample, said first chamber, said plunger having a valve in fluid communication with said first and second chambers.

55. (canceled)

56. A method of stimulating and stabilizing a biological sample comprising the steps of:
providing a sample collection container having a side wall, a bottom wall, and a closure member defining an internal compartment, said internal compartment having arranged therein: (i) a partition defining and fluidly separating first and second chambers in said internal compartment, said first chamber positioned in association with said closure member to receive said biological sample, said partition having a valve in fluid communication with said first and second chambers; and (ii) at least one stimulating agent contained within said first chamber in an amount effective to stimulate a biological sample, and at least one stabilizing agent contained within said second chamber in an amount effective to stabilize said biological sample; and
obtaining a biological sample and immediately thereafter introducing said biological sample into said first chamber, and mixing said biological sample with said at least one stimulating agent for a desired period of time so as to form a stimulated biological sample; and
stabilizing said stimulated biological sample immediately after said desired period of time by opening of said valve and mixing the contents of said first and second chambers.

57-64. (canceled)
